(19) United States
(12) Patent Application Publication
(10) Pub. No.: US 2004/0048346 A1
(43) Pub. Date: Mar. 11, 2004

(54) Synthesis of chiral intermediates useful in preparing pharmacologically active compounds

(76) Inventor: Raveendra Khandurao Tikare, Majiwada (IN)

Correspondence Address: Kit M Stetina, Stetina Brunda Garred & Brucker, Suite 250, 75 Enterprise, Aliso Viejo, CA, 92656, US

(21) Appl. No.: 10/416,171

(22) PCT Filed: Nov. 21, 2001
(86) PCT No.: PCT/IB01/02794

(30) Foreign Application Priority Data
Nov. 23, 2000 (GB) 0028523.9

(51) Int. Cl. C12P 13/00

(52) U.S. Cl. 435/128

(57) ABSTRACT

A process is disclosed for preparing (R)-2-hydroxy-4-phenylbutyronitrile of formula (I) wherein * signifies the (R) stereoisomer; and Ph is the phenyl group C6H5, which process comprises reacting, in a biphasic system, 3-phenylpropionaldehyde of formula (X): with a cyanide compound in the presence of (R)-hydroxynitrilase, wherein the reaction is carried out a temperature below (10)° C. Preferably, the reaction is carried out at a temperature in the range of from −(5)° to (0)° C. The compounds of formula (I) thereby prepared are useful in the preparation of the family of ACE inhibitors known as ‘prils’, of the general formula (A): wherein R′ is hydrogen or C1-C2 alkyl and R″ is selected from a large number of possible moieties. Example of “prils” include lisinopril, cilazapril, enalapril, benazepril, ramipril, delapril, enalaprilat, imidapril, spirapril, trandolapril and others. These ‘prils’ compounds are chiral compounds, only one of their diastereomers being pharmacologically active. Use of a chiral intermediate (I) thereby avoids the necessity to isolate and purify the active ‘pril’ diastereomer, rather than using a racemic mixture, for pharmaceutical/medical applications.

The present invention relates to a process for the synthesis of chiral compounds and, in particular, chiral nitrites for use as intermediates in the synthesis of the family of ACE inhibitors known as ‘prils’.

The prils have the general formula (A):

Ph-CH₂-CH₂—CH(COOR′)—NH(R″) (A)

wherein R′ is hydrogen or C₁-C₂ alkyl and R″ is selected from a large number of possible moieties. Examples of “prils” include lisinopril, cilazapril, enalapril, benazepril, ramipril, delapril, enalaprilat, imidapril, spirapril, trandolapril and others.

These ‘pril’ compounds are chiral compounds, only one of their diastereomers being pharmacologically active. It is therefore necessary to isolate and purify the active diastereomer, rather than using a racemic mixture, for pharmaceutical/medical applications.

Typically, separation of diastereomers is carried out by preferential crystallisation, for example as described in U.S. Pat. No. 5,616,727. However, the yields from such crystallisations are often low and, indeed, the yield from the process used in U.S. Pat. No. 5,616,727 was only 68%.

Alternatively, a stereochemical synthesis may be used, wherein various intermediates used in the preparation of the ‘prils’ are, in turn, prepared in chiral form, which results in a predominance of the desired diastereomer in the final ‘pril’ product. However, such chiral syntheses are complex and the yields are unsatisfactory.

The present invention relates to an improved, stereospecific process for the synthesis of an intermediate for making ‘pril’ compounds. This intermediate can then be converted to the required ‘pril’ isomer, or any other desired end-product, without loss of stereospecificity.

One of the building blocks in the synthesis of the ‘prils’ is a cyanohydrin containing the common ‘pril’ moiety Ph-CH₂-CH₂—CH—, which cyanohydrin can then be converted, via the corresponding carboxylic acid ester, to the desired ‘pril’. As discussed by C G Kruse in “Chirality in Industry” (Ed. Collins et al, chapter 14 (1992)), it is probable that the use of enantiomerically pure cyanohydrins as building blocks for the production of chiral industrial chemicals will continue to grow. This avoids the problems associated with the optical resolution or asymmetric synthesis of certain products. New routes to homochiral cyanohydrins represent, therefore, an opportunity to enlarge the pool of chiral starting materials, which are available to the fine chemicals industry. Several criteria must be realized fully before the optically pure cyanohydrins can be adopted as raw materials for industrial processes. These are:

(i) the availability of a range of methods for the manufacture of cyanohydrins with a high enantiomeric excess (ee) in an economically feasible way;

(ii) the preservation of optical purity during subsequent chemical transformations; and

(iii) the possibility of chirality transfer by diastereoselective reactions at either the cyano group or the main organic residue.

A method that has been proposed for the preparation of optically active cyanohydrins, which are useful in the preparation of, inter alia, the optically active ‘prils’ of formula (A) above, involves synthesis of (R)-2-hydroxy-4-phenyl butyronitrile (I):

* Ph-Ch₂-Ch₂—CH(OH)—CN (I)

wherein * signifies the (R) stereoisomer; and Ph is the phenyl group C₆H₅.

This method has been reported in U.S. Pat. No. 5,008,192 (and European patent specification no. 326 063), in which the reaction between an aldehyde and hydrogen cyanide is carried out in a homogeneous aqueous medium comprising oxynitrilase at a temperature varying from −5 to +50° C. and a pH value ranging from 4 to 6.5. Using this method, the nitrile (I) is said to be produced in a chemical purity of up to 93.8% and an optical purity of 95.1%. According to this US patent specification, however, “ . . . since the enzyme activity is considerably reduced by the presence of even small amounts of organic co-solvents (for example ethanol), the process should be carried out in the substantial absence of an organic co-solvent”. Thus, it strongly recommends the avoidance of any organic co-solvents in the reaction. There is no mention, however, of the possibility of the use of water-immiscible solvents, thereby signifying that biphasic reactions are also to be avoided.

Another method involves the use of the stereospecific enzyme (R)-hydroxynitrilase (also known as (R)oxynitrilase) in a two-phase reaction. For example, European patent specification no. 547 655 describes the reaction of phenylpropionaldehyde with hydrogen cyanide (HCN) at 10° C. and pH 4.5 in the presence of pure (R)-hydroxynitrilase at a concentration of 1.5 mg enzyme per mmol of aldehyde and in the presence of a buffer. This specification reports that this process resulted in an enantiomeric excess of the corresponding (R)-cyanohydrin of formula (I) hereinabove of “ca. 90” (optical purity ca 90%).

In the same example, this European patent specification discloses up to 99% enantiomeric excess when applying similar reaction conditions to other substrates, but clearly the reaction is much less successful in the case of the production of (R)-2-hydroxy-4-phenylbutyronitrile (I). If, therefore, one were to use the process of European patent specification no. 547 655 to prepare the ‘pril’ intermediate of formula (I), further purification would be required in order to provide the level of enantiomeric excess (ee) of the (R) isomer that is desired (ie, an ee of at least 97-98%). As mentioned above, such purification is a costly process, especially on a production scale, using chromatographic separation. Furthermore, this additional step reduces the yield of (R) isomer. High initial purity is therefore required in the preparation of (R)-2-hydroxy-4-phenylbutyronitrile (I) for it to be commercially advantageous in the synthesis of ‘prils’.

We have therefore looked at the possibility of using alternative methods of synthesizing this nitrile, but none of these appeared to provide the desired combination of high ee (eg 97-98%); economic reaction time; acceptable yields (eg 95-97%); and overall ease of handling and commercial viability of the process.

Instead, we have surprisingly found that, by careful selection of novel reaction conditions, we can obtain the desired ee in high yields and under commercially-acceptable conditions, using the two-phase oxynitrilase process.

Accordingly, the present invention provides a process for preparing (R)-2-hydroxy-4-phenylbutyronitrile of formula (I), which comprises reacting, in a biphasic system, 3-phenylpropionaldehyde of formula (X):

Ph-CH₂-CH₂—CHO (X)

with cyanide compound in the presence of (R)-hydroxynitrilase, wherein the reaction is carried out a temperature below 10° C.

The biphasic system comprises (i) an aqueous phase comprising an aqueous solution of the enzyme and (ii) an organic phase comprising a solution of the cyanide compound and the aldehyde (X) in a water-immiscible organic solvent. The aqueous phase may also comprise a pH-controlling buffer, and some cyanide compound may also be present in the aqueous phase, as will be described later. The reaction of the aldehyde of formula (X) with the cyanide compound takes place in the organic phase.

In the process according to the invention, the cyanide compound is preferably hydrogen cyanide.

The reaction is suitably carried out at a temperature below 5° C., preferably below 0° C. In a particularly preferred process, the reaction is carried out at a temperature in the range of from −5° to 0° C.

The reaction may be carried out over a wide range of pressures, but is preferably carried out at atmospheric pressure.

The process is suitably carried out such that the concentration of the nitrilase is greater than 1.5 mg per mmol of the aldehyde (X), preferably at least 2 mg per mmol of the aldehyde (X). It is particularly advantageous to employ the nitrilase at a concentration in the range of from 2 to 2.2 mg per mmol of the aldehyde (X).

For optimum performance, the reaction is suitably carried out at a pH in the range of from 4.5 to 6, preferably at a pH in the range of from 5.4 to 5.6. The pH of the reaction is suitably maintained within the range specified above by using a buffering agent in an aqueous solution. Thus, the aqueous phase of the reaction preferably comprises a suitable buffering agent such as an acetate buffer, or a non-acetate buffer eg citrate, glutamate, succinate or phthalate, but preferably a citrate, such as an alkali metal citrate, eg sodium or potassium citrate.

If the concentration of the buffer is relatively low, it may cause the pH of the aqueous phase containing the enzyme to vary during any recycling of said aqueous phase and hence the pH may have to be adjusted after each cycle. However, if the concentration of the buffer is relatively high, this may result in emulsification of the reaction mixture, thereby making phase separation and subsequent work-up of the reaction mixture much more difficult. Therefore, buffer is suitably used in a concentration in the range of from 0.3 to 1 Molar, preferably from about 0.4 to 0.6 Molar, eg about 0.5 Molar.

Using the specific novel conditions, particularly of temperature and enzyme concentration, and especially temperature but also pH, described herein, it has surprisingly been found that an enantiomeric excess (ee) of the (R) isomer of formula (I) of >98% can be achieved, with a yield also of >98% of theoretical yield, by weight.

In the process of the present invention, the ratio of the volumes of the aqueous phase to the organic phase is suitably in the range of from 1:5 to 5:1, and it is important to control the concentration of the cyanide compound in the organic phase. This is because HCN (the cyanide compound) is miscible in both phases. Even though it is soluble in the organic phase, its solubility in the aqueous phase is greater. For instance, if the volume of the organic phase is increased, nevertheless keeping the strength of the cyanide compound (eg hydrocyanic acid) constant, the reaction will remain substantially unaffected. However, if the volume of the organic phase is increased by diluting the concentration of the cyanide compound in said phase, the rate of reaction will be considerably slower. The strength of the cyanide compound in the organic phase is suitably in the range of from 6 to 6.5% weights by volume (eg 6-6.5 g of cyanide compound per 100 ml of organic phase).

Again, by changing the volume of the aqueous phase, the concentration of the cyanide compound will change in the organic phase; accordingly, if the volume of the aqueous phase is increased, the relative strength of the cyanide compound in the organic phase will decrease, which will—in turn—decrease the rate of the reaction.

Particularly preferred is when the cyanide compound is HCN, generated in situ by reaction of alkali metal cyanide, such as potassium or sodium cyanide, with a mineral acid, such as hydrochloric acid.

Most preferably, the HCN is prepared in an organic solvent to avoid handling the HCN itself and so that it is ready for use in the enzyme reaction, which itself requires an organic solvent for the organic phase of the reaction.

Suitable organic solvents include those described in European patent specification no. 547 655 for the purpose, namely: di-(C₁-C₆)alkyl ethers, (C₁-C₅)carboxylic (C₁-C₅)alkyl esters, di-(C₁-C₅)alkyl ketones, (C₄-C₈)aliphatic alcohols, and mixtures of these solvents with each other or with nonpolar diluents. Preferred examples of such water-immiscible solvents are: diethyl ether, di-n-propyl ether, di-isopropyl ether, di-n-butyl ether, di-isobutyl ether, methyl-t-butyl ether, ethyl acetate, n-propyl acetate, isopropyl acetate, isomeric butyl acetates, isomeric amyl acetates, methylethylketone, diethylketone, and methylisobutylketone. Suitable examples of non-polar diluents are aromatic hydrocarbons, aliphatic hydrocarbons and chlorinated aromatic or aliphatic hydrocarbons, such as toluene, xylene, hexane, cyclohexane, trichloroethene or chlorobenzene.

Preferred solvents are ethers and alcohols, especially dialkyl ethers and particularly di-isopropyl ether.

It is preferred that the molar ratio of the 3-phenylpropionaldehyde (X) to the cyanide compound in the reaction is in the range of from 1:1 to 1:6, preferably at least 1:3.

Another surprising advantage of this invention is that the aqueous phase comprising the nitrilase can be recycled for use in subsequent reaction(s) to a higher order than when using the conditions disclosed in European patent specification no. 547 655. This describes only triple recycling when a benzaldehyde is the substrate, but recycling would be even less successful under such conditions if propionaldehyde were the substrate. This is due to the fact that under the reaction conditions of this European patent specification, the chemical reaction competes with the enzymatic reaction resulting in low enantiomeric purity; moreover, this latter reaction causes loss of enzyme activity thereby reducing the number of cycles that can be performed. By contrast, we find that, using the novel conditions of the present invention, excellent results are still obtained after recycling the aqueous enzymatic phase at least ten times, eg twelve times, achieving an ee of at least 97%.

The present invention therefore further provides (R)-2-hydroxy-4-phenylbutyronitrile (I) whenever prepared by a process according to this invention; and such a compound (I) for use in, or whenever used in, the preparation of a stereospecific ‘pril’ of formula (A). Furthermore, there is provided a method for the preparation of a stereospecific ‘pril’ of formula (A), which method comprises preparation of (R)-2-hydroxy-4-phenylbutyronitrile (I) by a process according to this invention; and a stereospecific ‘pril’ of formula (A), whenever prepared by such a process.

This invention will now be illustrated by reference to the following non-limiting Examples.

Description A: Preparation of Hydrocyanic Acid in Di-Isopropyl Ether

A 1 litre 3-necked flask, equipped with a mechanical stirrer (Teflon™ gland), dropping funnel and internal thermometer pocket, was charged with sodium cyanide granules (52 g, 1.06 moles). 50 ml water was added, stirred and then 300 ml diisopropyl ether added. The mixture was stirred vigorously and the temperature brought down to 0°-5° C. 5N HCl (188 ml) was added drop-wise at 0°-5° C. (≈1½ hr) to sodium cyanide solution until the pH of the solution was 5.4 (the last 2-3 ml was added carefully). The reaction mass was taken into a 1 litre separating funnel. The aqueous layer was separated and carefully destroyed by sodium hypochlorite solution. Di-isopropyl ether fractions were collected in a 500 ml amber-coloured bottle and stored in a freezer.

### EXAMPLE 1

### Preparation of (R)-2-Hydroxy-4-Phenyl Butyronitrile

To a solution of 3-phenylpropionaldehyde (50 g, 0.37 mole) in di-isopropyl ether, was added 250 ml citrate buffer (pH 5.4, 0.5M, 5×3-phenylpropionaldehyde). The solution was cooled to 0° C. Oxynitrilase enzyme extracted from almonds was added (2000 units, ie 16.39 mg, per gram of 3-phenylpropionaldehyde) and 6-7% HCN solution prepared according to Description A (30.2 g, 1.12M) in di-isopropyl ether. The mixture was stirred for 30 minutes, having an aqueous:organic phase ratio of 1:2 by volume. The organic phase was separated and concentrated under reduced pressure to yield 98% theoretical yield by weight of the title compound with enantiomeric excess of 98%.

### EXAMPLE 2

### Preparation of (R)-2-Hydroxy-4-Phenylbutyronitrile by Recycling

The aqueous phase of the reaction from Example 1 was added to a solution of 3-phenylpropionaldehyde solution in di-isopropyl ether at a temperature in the range of from −5 to 0° C. 10% extra oxynitrilase enzyme extracted from almonds was added, followed by the 6-7% HCN solution in di-isopropyl ether. By this is meant that 10% of oxynitrilase enzyme in units was added in each cycle above the total enzyme charged initially, sc that when initially 2000 units of enzyme were used, a further 200 units of enzyme was charged for each and every cycle. The mixture was stirred for 30 minutes, then worked up as described in Example 1 to yield 98% of the title compound with enantiomeric excess of 98%. The enzyme was re-cycled ten times, resulting always in 98% of theoretical yield by weight of the title compound with enantiomeric excess of 98%.

Summary of Examples 1 & 2: (R)-2-Hydroxy-4-Phenyl Butyronitrile

| | | | Ratio of | | | | | ee |
|---|---|---|---|---|---|---|---|---|
| | | HCN | Aqueous: | Reaction | Enzyme | Reaction | Yield | (%) |
| Substrate | pH | Strength | Organic | Temp. | Conc.^{a} | Time | % | HPLC |
| 3-phenyl | 5.4 | 6.5% | 1:2 | −5-0° C. | 2-2.2 | 30 mins | 98 | 98 |
| propion- | | | | | | | | |
| aldehyde | | | | | | | | |

Note:
^{a}Enzyme concentration was calculated as follows:
Enzyme concentration = Enzyme in mg/Aldehyde in mmol
Enzyme 122 units = 1 mg
1 g of 3-phenylpropionaldehyde (MW = 134) = 7.46 mmoles
Enzyme for 1 g of 3-phenylpropionaldehyde = 2000 units = 16.39 mg
Enzyme concentration = 16.39/7.46 = 2.19

Spectral Data:

1. IR: OH 3400 cm⁻¹-3500 cm⁻¹; CN 2250 cm⁻¹

2. NMR: (CDCl₃, TMS) 7.3 (s, 5H), 4.4 (t,1H), 3.8-4(bs, 1H), 2.7-3 (q, 2H), 2-2.3 (q, 2H)

3. HPLC: Column: CHIREX -3014

Phase description: (S)-Valine and (R)-1-α-Naphthyl ethylamine

Bond type: covalent 250×4.6 mm

Mobile phase: Hexane:Dichloroethane:Ethanol:Acetic acid=500:150:5:0.6;

Flow rate: 1 ml/min; Wave length: 254 nm

Retention time: (R)-isomer=23.06 min; (S)-isomer=24.02 min

4. TLC: Silica gel; Acetone:Hexane 15:85; R_{f}=0.30

1. A process for preparing (R)-2-hydroxy-4-phenylbutyronitrile of formula (I):
* Ph-CH₂-CH₂—CH(OH)—CN (I)
wherein * signifies the (R) stereoisomer; and Ph is the phenyl group C₆H₅,
which process comprises reacting, in a biphasic system,
3-phenylpropionaldehyde of formula (X):
Ph-CH₂-CH₂—CHO (X)
with a cyanide compound in a water-immiscible organic solvent in the presence of an aqueous solution of (R)-hydroxynitrilase,
wherein the concentration of the nitrilase in the aqueous phase is greater than 1.5 mg per mmol of the aldehyde (X) and the reaction is carried out at a pH in the range of 4.5 to 6 at a temperature below 10° C.

2. A process according to claim 1, wherein the reaction is carried out at a pH in the range of from 5.4 to 5.6.

3. A process according to claim 1 or claim 2, wherein the concentration of the nitrilase is in the range of from 2 to 2.2 mg per mmol of the aldehyde (X).

4. A process according to any preceding claim, wherein the reaction is carried out at a temperature below 5° C.

5. A process according to any preceding claim, wherein the reaction is carried out at a temperature below 0° C.

6. A process according to any preceding claim, wherein the reaction is carried out at a temperature in the range of from −5° C. to 0° C.

7. A process according to any preceding claim, wherein buffer is used in a concentration in the range of from 0.3 to 1 Molar.

8. A process according to any preceding claim, wherein buffer is used in a concentration in the range of from about 0.4 to 0.6 Molar.

9. A process according to any preceding claim, wherein the cyanide compound is hydrogen cyanide.

10. A process according to any preceding claim, wherein the ratio of the volumes of the aqueous phase to the organic phase is in the range of from 1:5 to 5:1.

11. A process according to any preceding claim, wherein concentration of the cyanide compound in the organic phase is in the range of from 6 to 6.5% weight by volume (eg 6 to 6.5 g of cyanide compound per 100 ml of organic phase).

12. A process according to any preceding claim, wherein the cyanide compound is HCN, generated in situ by reaction of alkali metal cyanide, including potassium of sodium cyanide, with a mineral acid, including hydrochloric acid.

13. A process according to any preceding claim, wherein the cyanide compound is prepared in an organic solvent.

14. A process according to any preceding claim, wherein the cyanide compound is prepared and/or reacted in an organic solvent selected from ethers and alcohols, including dialkyl ethers (eg di-isopropyl ether).

15. A process according to any preceding claim, wherein the molar ratio of the 3-phenylpropionaldehyde (X) to the cyanide compound in the reaction is in the range of from 1:1 to 1:6, including at least 1:3.

16. A process according to any preceding claim, wherein the nitrilase is recycled at least ten times.

17. A process according to any preceding claim, whereby the (R)-2-hydroxy-4-phenylbutyronitrile is prepared to an ee of at least 97%.

18. The compound (R)-2-hydroxy-4-phenylbutyronitrile (I) whenever prepared by a process according to any preceding claim.

19. The compound (R)-2-hydroxy-4-phenylbutyronitrile (I) for use in, or whenever used in, the preparation of a stereospecific ‘pril’ of formula (A):
Ph-CH₂-CH₂—CH(COOR′)—NH(R″) (A)
wherein R′ is hydrogen or C₁-C₂ alkyl and R″ is an organic moiety.

20. A method for the preparation of a stereospecific ‘pril’ of formula (A), which method comprises preparation of (R)-2-hydroxy-4-phenylbutyronitrile (I) by a process according to any of claims 1 to 17.

21. A stereospecific ‘pril’ of formula (A), whenever prepared by a process according to any of claims 1 to 17.

22. A process, compound or use according to any preceding claim, as hereinbefore described, with particular reference to the Examples.
